(19) United States
(12) Patent Application Publication
(10) Pub. No.: US 2021/0260311 A1
(43) Pub. Date: Aug. 26, 2021

(54) METHODS, DEVICES, KITS AND SYSTEMS FOR DELIVERY OF LARGE VOLUME OF PRESSURIZED GAS BY INHALATION

(71) Applicants: Yissum Research Development Company of the Hebrw University of Jerusale Ltd., Jerusalem (IL); Hadasit Medical Research Services and Development Ltd., Jerusalem (IL)

(72) Inventors: Tomer BATASH, Lod (IL); Sigal SHAFRAN-TIKVA, Jerusalem (IL); Avner EHRLICH, Jerusalem (IL); Yaakov NAHMIAS, Mevaseret Zion (IL); Amnon BUXBOIM, Tel Aviv (IL); Yaron BLINDER, Haifa (IL); Yoav MINTZ, Jerusalem (IL); Elchanan FRIED, Jerusalem (IL); Dan ORBACH, Nir Banim (IL)

(21) Appl. No.: 17/254,103

(22) PCT Filed: Jun. 20, 2019
(86) PCT No.: PCT/IL2019/050698

§ 371 Date: Dec. 18, 2020

Related U.S. Application Data
(60) Provisional application No. 62/687,394, filed on Jun. 20, 2018.

(51) Int. Cl. A61M 15/00; A61M 13/00; A61M 11/00; A61M 16/06

(52) U.S. Cl. CPC A61M 15/0095; A61M 13/00; A61M 15/0021; A61M 16/06; A61M 11/003

(57) ABSTRACT

There are provided herein methods, devices, kits and systems utilizing respiratory mask for delivering pressurized fluid to a subject via inhalation in an efficient manner. The fluid may include gas and/or drug and by utilizing the methods, devices, kits and systems provided, efficient drug delivery to the subject's airways is achieved. The systems, devices, kits and methods further allow inducing insufflation/exsufflation in particular in subjects having impaired suffering from low neuromotor capacity, such as spinal cord injuries (SCI) patients.

### TECHNICAL FIELD

The present disclosure relates generally to methods, devices, kits and systems utilizing respiratory masks for delivering pressurized air/aerosol to a subject via inhalation.

### BACKGROUND

Respiratory masks are commonly used in a wide range of applications, including for medical treatment. In particular, masks are typically used for gas administration or to provide a continuous positive air pressure to a patient to assist in breathing. Furthermore, masks have been increasingly used for drug delivery, particularly for treating respiratory disorders such as asthma, accumulation of mucus, respiratory infections, etc.

One common use for respiratory masks is insufflation of patients with spinal cord injury (SCI), other neuromuscular deficiencies and other respiratory diseases. These patients are unable to clear their respiratory secretions due to reduced respiratory capacity and ineffective cough.

In addition, the compliance of the lungs is decreased in these patients, further limiting their ability to insufflate air into the lungs, thus limiting their respiratory capacity and other activities that require variation in respiration (i.e. coughing, yelling, etc.) or increased respiratory demand (aerobic activity).

Inducing an effective cough requires rapid release of high volume of air in order to create enough pressure-force to shear the mucus and other disturbances. Spinal cord injury (SCI) and other neuromuscular deficient (NMD) patient are unable to create an effective cough due to a weakened costal muscle and diaphragm activity. These patients have low Forced Vital Capacity (FVC), a high Residual volume and Forced Residual Volume (RV/FRV) and low Forced Expiratory Volume (FEV1). Altogether, such unfavored pulmonary function cause a low air shear-force to be induced by a forced exhale or coughing, reducing the capability of these patients to remove mucus and other disturbances and/or refresh their lungs effectively.

For example, healthy subjects capable of coughing effectively have on average a FVC of about 4-5 L, a FEV1 of about 3-4 L/s and a FRV of about 1-2 L. In comparison, patients with tetraplegia have only about 40-70% of the FVC, about 45-75% of the FEV1 and about 110-160% of the FRV of an uninjured patient. Essentially, this means that paralyzed patients exhale approximately 17% the air volume of a healthy patient (0.5 L in comparison to the 3 L exhaled by an uninjured subject), at a much lower speed, producing a significantly lower shear-force on pulmonary disturbances and producing an ineffective cough. Even patients with paraplegia, which have better pulmonary functions, fall as low as 70% FVC and FEV1 and up to 150% FRV, exhaling less than a half than an uninjured subject would, at a lower speed, producing an insufficient or ineffective cough.

In terms of expiratory flows, effective coughs require Peak expiratory/coughing flow (PEF/PCF) of over about 160 L/min, a difference of over about 20 L/min between the peak expiratory flow and the peak inspiratory flow (PEF-PIF), and/or a ratio bellow about 0.9 between the peak inspiratory flow and the peak expiratory flow (PIF/PEF).

Mechanical and manual devices of insufflation and exsufflation are based on compression of a chamber (either in the machine or in the air bag), and therefore, when the volume delivered surpass the chamber's volume, the action is done in lags. Inhalation and exhalation are short processes (0.5-6 seconds), and delay in the process is limiting the volume capable of being delivered and its efficacy, due to limitation in the pressure (and thereby flow) safe for inhalation. Most currently used devices in order to produce an effective secretion removal require the assistance of a medical professional (doctor, nurse, physiotherapist, etc.) or a trained caregiver. In cases where self-management is desired, an effective secretion removal requires high expertise and substantial manual performance of the user.

In other cases, respiratory masks are used for pulmonary delivery. The most common pulmonary delivery platforms are inhalers and nebulizers, such as in the case of bronchodilators (acute asthma treatment) and mucolytics. These platforms administer small volume of concentrated aerosolized drug into the oral cavity, later to be induced by the patient's own inhalation. This method of delivery causes most drug to be delivered in the initial volume of inhalation and therefor, to be absorbed in the upper pulmonary tubing. Nevertheless, most common inhalers require the patient to coordinate his own inhalation with the administration of the drug. Other solutions, such as incorporating the inhaler within a ventilator does not solve the delivery issue, and possess difficulties of its own, either by accessibility (as most ventilators are power operated and stationary) or by additional complexity (such as pressing against an air bag simultaneously with releasing the drug). Administration of medication, using inhalation devices, can have lower undesired side effects and on-site activity. However, most treatment regimens have medium to low compliance and effectivity due to limited accessibility of the drugs to their target site and the high complexity required for effective administration.

There is thus a need in the art for improved and easy to use devices and systems utilizing respiratory masks for medical purposes, for example, for patients suffering from SCI or other neuromuscular deficiencies, to improve their breathing capabilities in general and to improve effective removal of various disturbances from their lungs.

### SUMMARY

Aspects of the disclosure, according to some embodiments thereof, relate to a platform for delivering pressurized gas, such as air or aerosol to a subject, via inhalation. In some embodiments, the methods, devices, kits and systems (platforms) disclosed herein can be used to improve breathing, increase lung compliance, pulmonary functions and airway clearance, in particular in subjects having neuromuscular deficiencies affecting their breathing, or patients having lung disorders, by providing pressurized gas to these patients. In some embodiments, the methods, devices and systems disclosed herein can advantageously induce insufflation or insufflation/exsufflation regimes in such patients, by actively introducing pressurized gas into the pulmonary system. Such active delivery of gas (such as, air), in particular in a higher-then-normal volume of air, enables in some embodiments to remove secretions in airways of the patients, by a passive cough, secretions that otherwise could not have been removed by the patients. In some embodiments, the pressurized gas delivered by the devices and systems disclosed herein may further include a drug. In some embodiments, the pressurized fluid may be delivered with the aid of dedicated mask(s).

According to some embodiments, the device disclosed herein is based on a portable pressurized air source that can induce insufflation or insufflation/exsufflation regimes by actively introducing pressurized gas (such as, air) into the pulmonary system. Such active delivery of air, in particular in a higher-then-normal volume of air, enables in some embodiments to ventilate the airways of a user. In some embodiments, the platform may use pressurized gas to extract gas in accordance with the Bernoulli law.

In some embodiments, hyperinflation (i.e., delivery of gas, such as, air, in higher-then-normal volumes) is advantageous for serval reasons: First, actively-delivered-air can access most sites/regions in the pulmonary tubing; even the lower lungs, and up to the alveoli itself; to allow the air to accumulate under the mucus and other disturbances. This accumulation can assist with detaching or disturbing these disturbances and thereby, assist with their removal. Additionally, actively-delivered-air supports improvements in lung compliance, oxygenation, and may further benefit intubated and mechanically ventilated critically ill patients. Further, in some embodiments, the gas may include a drug, such as in the form of an aerosol and such treatment may be used, for example, for site specific pulmonary delivery of a drug.

According to some embodiments, the platforms disclosed herein are advantageously designed to enable self-management and use by inhaling through a dedicated respiratory mask, such as, in the form of a mouthpiece having a biting surface, such that by biting and gripping the surface of the mouthpiece by the teeth, the mask is secured in the subject's mouth and a tight seal is achieved, such that the gas can advantageously flow directly into the subject's mouth without any risk of leakage. In accordance with some embodiments, such design enables using the dedicated respiratory mask with minimal hand-assistance. In some embodiments, the respiratory mask may be a face mask which further includes a chin support assembly for mounting the mask on the subject's face. Thus, advantageously, the respiratory mask is configured for self-use, also among subjects suffering from low neuromotor capacity, such as spinal cord injuries (SCI) patients.

Mechanical and manual devices of insufflation and exsufflation are based on compression of a chamber (either in a machine or in an air bag), and therefore, when the volume that is delivered surpass the chamber's volume, the action should be done in lags, which lowers the efficiency of the process. Inhalation and exhalation are short processes (0.5-6 seconds) may delay in the process is limiting the volume capable of being delivered and its efficacy, due to limitation in the pressure and flow that are safe for inhalation. Thus, advantageously, as disclosed herein, insufflation and exsufflation mediated by delivery of gas from a pressurized source ensures continuous air flow, even if larger volumes (0.75-2 L) are delivered.

According to some embodiments, when utilizing the methods, devices and systems disclosed herein, the pressurized gas (for example, air and/or aerosolized drug) is actively delivered into the subject's airways, for example, to the subject's mouth, in a predetermined volume (that may include, for example, a drug), delivering a predetermined dose. In addition, in such embodiments, because the gas is delivered directly into the subject's mouth, the eyes of the subject and the environment are not exposed to the gas even if the respiratory mask is not completely sealed. Thus, the risk of damage to the eyes (such as developing cataracts as a result of aerosolized drugs), loss of material and environmental contamination (such as exposure of companions and caregivers to drug leakage) is mitigated.

According to some embodiments, the respiratory mask may be disposable and/or recyclable and could further act to absorb, contain and dispose of exerted mucus and other disturbances, as it may be discarded after the use. This may eliminate the need for cleaning and sterilization, as well as highly reduce the risk of repeated-use-associated infections that other devices hold. In other embodiments some or all parts of the respiratory mask may be reused several times before disposal.

According to some embodiments, the devices and systems (platforms) disclosed herein, allow efficient and safe administration of a drug. In some embodiments, the drug may be aerosolized prior to administration in a large volume of pressurized gas. In this manner, the concentration of the drug per volume is low and the drug may be delivered further than the gas's initial impact site. This advantageous property usually cannot be achieved by mechanical ventilators or inhalers used in the art. According to some embodiments the aerosolization can occur before the assembly of the mask and the aerosol is be stored in the pressurized reservoir.

According to some embodiments, the platforms disclosed herein, are configured to actively deliver the gas (with or without drug) to most target pulmonary sites and/or allows delivery of gas even when the patient is unable to efficiently inhale on its own (for example, in the case of an acute asthma incidence). This may also be applied in pulmonary administration of medication to other drugs and treatments, as it provides access to pulmonary sites that are less accessible by other methods of delivery.

According to some embodiments, the method of administration, that is, release of a small dose of drug over the duration of insufflation may be used in dose-reduction of immediate affecting drugs (such as bronchodilators). When reaching the desired effect (for example, opening of the medium and large airways in the lungs and restored breathing capability), administration may be stopped, thereby reducing side effects and minimize reduction in sensitivity, associated with hyper-dosing.

According to some embodiments, the use of the devices and systems utilizing the respiratory mask enables a controllably cumulative dosing which is advantageous for pulmonary delivery of a drug having an immediate effect or indications of effectiveness, such as, for example, capsaicin or Ventolin. This way of drug administration may reduce adverse effects and overdosing events, because patients could adjust and control drug dosage by stopping administration upon achieving a desired effect. For example, in some embodiments, administration of Salbutamol via the respiratory mask, in order to induce opening of the medium and large airways in the lungs and restore breathing capability, prevents drug overdose since the dosing ceases upon inhalation by the subject.

According to some embodiments, such mask/platform may be applied for the cumulative dosing of capsaicin via the respiratory mask to accurately assess and monitor the cough insensitivity, relevant to patients having with a nervous system disorder or injury, such as central nervous system (CNS) disorders, cervical injuries and/or spinal cord injuries (SCI) since the concentration needed to induce coughing indicates the disorder or severity of injury.

According to some embodiments, using the platforms disclosed herein, may advantageously facilitate a deeper delivery of a gas (such as air and/or aerosol) into the subject's respiratory system, including the lungs, lower and upper respiratory tracts and pulmonary alveoli. Thus, active delivery of a large volume of pressurized gas (such as air and/or aerosol) directly to the lungs is advantageous particularly to subjects having high residual volume of air in the lungs and/or low respiratory capacity. In addition, active targeted drug delivery reduces loss of drug and thus reduces adverse effects.

According to some embodiments, there are provided systems, devices and methods utilized to deliver pressurized gas via inhalation by a subject. In some embodiments, the pressurized gas includes a drug. The drug may include a pulmonary drug. Advantageously, activation by inhalation minimizes the risk of air leaking into the gastrointestinal track (the action of inhalation reflexively moves the epiglottis to seal the esophagus).

According to some embodiments, there is provided a device for delivering pressurized gas to a subject, the device comprising:

a tank holding a reservoir of pressurized gas;

a pressure and/or flow regulating element, interacting with the pressurized gas tank, said regulating element comprises a valve unit configured to controllably release the pressurized gas from the pressurized gas reservoir; and a first adaptor, located on the pressure regulating element, configured to fluidly connect to a corresponding adaptor of an external mask configured to interact with airways of the subject, wherein the first adaptor allows passage of the pressurized gas from the pressurized gas reservoir to the mask adaptor, only when the valve unit is open.

According to some embodiments, the valve unit may be opened by inhalation of the subject, to thereby allow the pressurized gas to be released from the pressurized gas reservoir and forced into the subject airways, via the first adaptor, the mask adaptor and the external mask.

According to some embodiments, the regulating element of the device may be further configured to regulate the pressure of the gas release from the pressurized gas reservoir into the subject airways.

According to some embodiments, wherein the valve unit comprises a deformable membrane configured to deform upon inhalation by a subject, to thereby allow opening of gas passages located between the pressurized gas reservoir and the first adaptor, to allow pressurized gas movement from the pressurized gas reservoir, through the valve unit, to the first adaptor.

According to some embodiments, the valve unit may include a deformable membrane configured to deform upon manual deformation by a subject, to thereby allow opening of gas passages located between the pressurized gas reservoir and the first adaptor, to allow pressurized gas movement from the pressurized gas reservoir, through the valve unit, to the first adaptor.

According to some embodiments, the valve unit may include a coupling element connecting the deformable membrane to a sealing unit, configured move the sealing unit upon deformation of the membrane by a subject, to thereby allow opening of gas passages located between the pressurized gas reservoir and the first adaptor, to allow pressurized gas movement from the pressurized gas reservoir, through the valve unit, to the first adaptor. In some embodiments, the coupling element may include such elements as, but not limited to: levers, strings, or any suitable type of coupling units, configured to open the sealing units in accordance to deformation of the membrane.

According to some embodiments, the valve unit may further include one or more closing units, configured to return of the sealing caps and allow closing of the gas passages.

According to some embodiments, the valve unit may further include one or closing units, that include such units as, but not limited to: cables, springs, strings, motors, or any suitable type of motion units, configured to return to their formation/tension and return to their previous state after they have been deformed/contracted.

According to some embodiments, the valve unit may further include one or more resistance units, configured to resist the return of the sealing caps and allow longer duration of gas passages. In some embodiments, allowing a flow duration of more than about 0.5 seconds. In some embodiments, allowing a flow duration of more than about 0.7 seconds. In some embodiments, allowing a flow duration of more than about 0.9 seconds. In some embodiments, allowing a flow duration of more than about 1 seconds.

According to some embodiments, the valve unit may further include one or more resistance units, that include such units as, but not limited to: springs, dashpots, electrical resistance units, or any suitable type of resistance units, configured to resist a change in their formation/tension and return to their previous state after they have been deformed/contracted.

According to some embodiments, the gas may include a drug. According to some embodiments, the regulator element may further include an aerosol chamber configured to aerosolize the drug.

According to some embodiments, the aerosol chamber may further include a particle size filter figured to determine the aerosolized particle size.

According to some embodiments, the drug may be in the form of a solution, gel, fine solid particles or gas.

According to some embodiments, the drug is configured for administration to the respiratory system of the subject.

According to some embodiments, the drug is selected from a group consisting of: an anti-inflammatory drug, a corticosteroid, a respiratory drug, a cough inducing drug, an anti-microbial drug, an anti-viral drug, an anti-fungi drug, chemotherapy, immunotherapy, an anti-cancer drug, coagulants, fluid permeability increasing drugs or any combination thereof.

According to some embodiments, the drug may be used for treating respiratory infections, excessive secretions, asthma, bronchospasm, bronchiectasis, lung cancer, chronic obstructive pulmonary disease (COPD), shortness of breath, airway and pulmonary bleeding, pleural effusion or any combination thereof.

According to some embodiments, the drug may be selected from a group of neuroactive substances selected from cannabis or any of its components, or any combination of neuroactive and pain mitigating substances thereof.

According to some embodiments, the drug may be used for recreational purposes or treating pain (including chronic pain), loss of appetite, depression, anxiety, post-traumatic stress disorder (PTSD), or any approved indication for neuroactive substances and or any combination thereof.

According to some embodiments, the drug comprises capsaicin.

According to some embodiments, the drug is used for inducing coughing in a subject.

According to some embodiments, the device may be used for inducing coughing in a subject suffering from spinal cord injury (SCI) or neuromuscular deficiencies

According to some embodiments, the device may be further configured to stop drug delivery once a cough is induced.

According to some embodiments, the pressurized gas tank may include a mechanism to allow the release of a volume of pressurized gas, when the valve is open.

According to some embodiments, the device may further include a sensor/responding element and a regulator.

According to some embodiments, the sensor/response element is configured to activate the regulator and elements upon inhalation, to allow gas flow (insufflation) from the pressurized gas tank to the first adaptor during inhalation.

According to some embodiments, the sensor/response element is configured to activate the regulator and elements upon exhalation/cough, to allow flow from the user's airway out (exsufflation).

According to some embodiments, the regulating element may further include a chamber fluidly connect to the first adaptor, said chamber is configured to facilitate pressure equilibration.

According to some embodiments, the tank may be configured to deliver gas volume and maintain a constant or predetermined rate of change in the gas pressure, such that mucus motility is outward of the user's airway.

According to some embodiments, the tank may include a floating/moving floor, deformable bag or any suitable unit that allows change in tank volume to maintain a constant or predetermined rate of change in the gas pressure, such that mucus motility is outward of the user's airway.

According to some embodiments, the tank may be configured to change its volume with moving elements that include, but not limited to: cables/springs/strings/motors, or any suitable type of motion units, configured to move at a predetermined rate to maintain a constant or predetermined rate of change in the gas pressure in the tank.

According to some embodiments, the tank may be configured to deliver gas volume, such that the volume delivered is about 100-600% (or any subranges thereof) of the user's vital capacity and at a range of about 0.5 Liter to about −3 L of gas

According to some embodiments, the tank may be configured to deliver and/or extract gas volume, such that the peak expiratory airflow (PEF) or peak coughing flow (PCF) is over about 160 L/min. in some embodiments, the peak expiratory airflow (PEF) or peak coughing flow (PCF) is over about 120 L/min. the peak expiratory airflow (PEF) or peak coughing flow (PCF) is over about 180 L/min

According to some embodiments, the tank may be configured to deliver gas volume and maintain a constant or predetermined rate of change in the gas pressure, such that the difference between the peak expiratory airflow (PEF) and the peak inspiratory airflow (PIF) is over about 20 L/min. In some embodiments, the difference between the peak expiratory airflow (PEF) and the peak inspiratory airflow (PIF) is over about 15 L/min. difference between the peak expiratory airflow (PEF) and the peak inspiratory airflow (PIF) is over about 25 L/min.

According to some embodiments, the tank may be configured to deliver gas volume and maintain a constant change in the gas pressure, such that a ratio between the peak inspiratory airflow (PIF) and the peak expiratory airflow (PEF), is lower than about 0.9. In some embodiments, the ratio between the peak inspiratory airflow (PIF) and the peak expiratory airflow (PEF), is lower than about 0.8. In some embodiments, the ratio between the peak inspiratory airflow (PIF) and the peak expiratory airflow (PEF), is lower than about 0.7.

According to some embodiments, the tank may be configured to maintain a constant or predetermined rate of change in the gas pressure, such that the maximal pressure insufflated is lower than about 75 cmH2O. In some embodiments, the maximal pressure insufflated is lower than about 65 cmH2O. In some embodiments, the maximal pressure insufflated is lower than about 85 cmH2O

According to some embodiments, the valve unit includes a positive expiratory pressure (PEP) one-way valve configured to prevent backflow, to thereby facilitate deeper drug delivery into the lungs of the subject and/or to increase hyperinflation capacity of the lungs.

According to some embodiments, the gas pressure in the pressurized fluid tank is between about 10-50 atm.

According to some embodiments, the pressurized gas tank may include an expanded collapsible bag having a fixed maximum dimension, and wherein the bag is used for storing the gas, such that the bag minimal volume defines the remaining effective/operational volume.

According to some embodiments, the device disclosed herein may be used for lung exercise, respiratory physiotherapy, to improve pulmonary functions, to increase lung compliance, to increase oxygenation or any combination thereof.

According to some embodiments, there is provided a kit for delivering pressurized gas to a subject, the kit comprising: a device as disclosed herein; and an external mask configured for use with said device.

According to some embodiments, the external mask in the kit may be selected from pharyngeal mask, laryngeal mask, face mask, endotracheal tube, or any combination thereof.

According to some embodiments, the mask is a pharyngeal mask, configured to be placed in the mouth of a subject to allow direct gas passage from the device, via a channel located on the mask adaptor of the pharyngeal mask, to the airways of the subject.

According to some embodiments, the pharyngeal mask may include a biting surface, configured to be placed in the oral cavity of the subject and to secure the mask in the pharyngeal cavity.

According to some embodiments, the mask of the kit is a face mask further comprising a chin support assembly to facilitate self-mounting of the mask on the subject's face with minimal hand-assistance.

According to some embodiments, the mask may further include a nose bridge for sealing the nose when said mask is worn.

According to some embodiments, the mask of the kit is disposable.

According to some embodiments, the mask of the kit is recyclable.

According to some embodiments, the external mask is further configured to absorb and/or store extracted mucus or other pulmonary disturbances released from the subject's airways.

According to some embodiments, there is provided a method for delivering pressurized gas to a subject, the method comprising: adjusting an external mask to the subject's face; and upon inhaling, triggering a release of pressurized gas from a device for delivering pressurized gas to a subject, the device comprising: a pressurized gas tank holding a reservoir of pressurized gas; a regulating element, interacting with the pressurized gas tank, said regulating element comprises a valve unit configured to controllably release the pressurized gas from the pressurized gas tank; and a first adaptor, located on the pressure regulating element, configured to fluidly connect to a corresponding adaptor of the external mask.

According to some embodiments, adjusting the mask may include placing the mask on the face of the subject and/or placing the mask within the subject's mouth.

According to some embodiments, the method may further include the step of collecting, storing or absorbing mucus or other pulmonary disturbances released or extracted from the subject's airways.

According to some embodiments, the method may aid in lung exercise, respiratory physiotherapy, to improve pulmonary functions, to increase lung compliance, to increase oxygenation or any combination thereof.

According to some embodiments, the method may aid in the increase of adherence for cough assisting, respiratory exercise or any combination thereof.

According to some embodiments, the method may aid in lowering acute pulmonary infections rates, preventing acute pulmonary infections, lowering acute pulmonary complication rates, preventing acute pulmonary complications, lowering chronic pulmonary complication rates, preventing chronic pulmonary complications or any combination thereof.

According to some embodiments, the method may aid in: treating of acute pulmonary infections, shorten the duration of acute pulmonary infections, treat acute pulmonary complication, shorten the duration acute pulmonary complications, treat chronic pulmonary complication, shorten the duration chronic pulmonary complications or any combination thereof.

According to some embodiments, there is provided a system for insufflation and exsufflation of pressurized gas to a subject, the system includes:

a respiratory mask configured to be adjusted on the face of the subject, and having an adaptor configured to fluidly connect to a pressurized gas tank via an insufflation port and to a venturi-based device via an exsufflation port; and

a sensor functionally associated with a regulator configured to trigger, upon the beginning of inhalation of the subject, insufflation of pressurized gas from the pressurized gas tank via the insufflation port and to the patients airways, wherein the system is further configured to induce exsufflation after a predetermined duration of a volume about equal or higher than the volume insufflated by directing the gas flow to the venturi-based device.

According to some embodiments, the respiratory mask used in the system may be selected from pharyngeal mask, laryngeal mask, face mask, endotracheal tube, or any combination thereof. According to some embodiments, the mask is a pharyngeal mask, configured to be placed in the mouth of a subject to allow direct gas passage from the device, via a channel located on the mask adaptor of the pharyngeal mask, to the airways of the subject. According to some embodiments, the pharyngeal mask may include a biting surface, configured to be placed in the oral cavity of the subject and to secure the mask in the pharyngeal cavity. According to some embodiments, the mask is a face mask further comprising a chin support assembly to facilitate self-mounting of the mask on the subject's face with minimal hand-assistance. According to some embodiments, the mask may further include a nose bridge for sealing the nose when said mask is worn. According to some embodiments, the mask is disposable. According to some embodiments, wherein the mask is recyclable. According to some embodiments, the external mask is further configured to absorb and/or store extracted mucus or other pulmonary disturbances released from the subject's airways.

According to some embodiments, the volume of the pressurized gas tank of the system may be about 50-500 mL or about 1-20 L.

According to some embodiments, the pressure of the pressurized gas in the pressurized gas tank of the system may be about 10-50 atm, or about 50-300 atm.

According to some embodiments, the gas used in the system may include a drug. According to some embodiments, the drug may be in the form of a solution, gel, fine solid particles or gas. According to some embodiments, the drug is configured for administration to the respiratory system of the subject. According to some embodiments, the drug comprises capsaicin. According to some embodiments, the drug may used for inducing coughing in a subject

According to some embodiments, the drug may be selected from a group consisting of: an anti-inflammatory drug, a corticosteroid, a respiratory drug, a cough inducing drug, an anti-microbial drug, an anti-viral drug, an anti-fungi drug, chemotherapy, immunotherapy, an anti-cancer drug, coagulants, fluid permeability increasing drugs or any combination thereof. Each possibility is a separate embodiment.

According to some embodiments, the drug may be used for treating respiratory infections, excessive secretions, asthma, bronchospasm, bronchiectasis, lung cancer, chronic obstructive pulmonary disease (COPD), shortness of breath, airway and pulmonary bleeding, pleural effusion or any combination thereof. Each possibility is a separate embodiment.

According to some embodiments, the drug may be selected from a group of neuroactive substances selected from cannabis or any of its components, or any combination of neuroactive and pain mitigating substances thereof. Each possibility is a separate embodiment.

According to some embodiments, the drug may be used for recreational purposes or treating pain (including chronic pain), loss of appetite, depression, anxiety, post-traumatic stress disorder (PTSD), or any approved indication for neuroactive substances and or any combination thereof. Each possibility is a separate embodiment.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale. In the Figures:

FIG. 1 schematically depicts a device for delivering pressurized gas to a subject, according to some embodiments;

FIGS. 2A-B schematically depict mechanism of storing and delivering pressurized gas (such as gas air and/or aerosol) under a constant or predetermined pressure gradient, according to some embodiments. FIG. 2A—a schematic cross section of a device for delivering pressurized gas showing the mechanism at the state of storing the gas in a reservoir. FIG. 2B—a schematic cross section of a device for delivering pressurized gas showing the mechanism at a state after at least part of the gas has been dispensed (delivered).

FIGS. 3A-C—schematically depict a mechanism of delivering pressurized gas via insufflation upon inhalation by a subject, according to some embodiments. FIG. 3A a schematic illustration of a perspective side view of the mechanism for regulating passage of pressurized gas through a valve. FIGS. 3B-3C schematic illustrations of top view of a cross section of a valve for regulating passage of pressurized gas in a closed mode (FIG. 3B) and open mode (FIG. 3C), wherein the valve is activated by inhalation of a subject;

FIGS. 4A-C—schematically depict a mechanism of delivering pressurized gas via insufflation upon inhalation by a subject and/or manual activation, according to some embodiments. FIG. 4A a schematic illustration of a perspective side view of the mechanism for regulating passage of pressurized gas through a valve. FIGS. 4B-4C—schematic illustrations of a top view of cross section of a valve for regulating passage of pressurized gas in a closed mode (FIG. 4B) and open mode (FIG. 4C), wherein the valve is activated by manual activation;

FIGS. 5A-B—schematically depict a mechanism of delivering aerosol, with a device for delivering pressurized gas via insufflation by a subject, according to some embodiments;

FIG. 6 schematically depicts a perspective view of an exemplary design of a pharyngeal mask (mouthpiece-based mask), according to some embodiments;

FIG. 7A schematically depicts a front view of a cup member of a face mask, according to some embodiments;

FIG. 7B schematically depicts a side view of the cup member of, according to some embodiments;

FIG. 8 schematically depicts a distal part of a cup member which includes a valve that ensures one-directional flow, according to some embodiments; and

FIG. 9 schematically depicts an automatic system of delivering and extracting pressurized fluid (insufflation-exsufflation) to a subject via a face mask, according to some embodiments.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

The following are terms which are used throughout the description and which should be understood in accordance with the various embodiments to mean as follows:

As used herein, the term “insufflation” is directed to the blowing of a gas to a body cavity. For example, the term insufflation includes pressurized delivery of gas via the respiratory tract, to the lungs.

The term “exsufflation” is directed to forcible breathing or blowing out from the respiratory tract. For example, the term exsufflation relates to clearing the respiratory tract by forcing air from the lungs.

The terms “mask” and “respiratory mask” may interchangeably be used. The terms encompass any type of means capable of accessing the airways of a subject (patient) that is wearing/using/holding/biting the mask, to allow direct gas transfer to/from the respiratory airways of the subject. In some embodiments, the mask may be selected from, but not limited to: pharyngeal mask, laryngeal mask, face mask, endotracheal tube, and the like, or any combination thereof. Each possibility is a separate embodiment. In some exemplary embodiments, the mask may be a face mask. In some exemplary embodiments, the mask is a pharyngeal mask. In some exemplary embodiments, the mask is a combination of a face mask and pharyngeal mask.

As used herein, the term “gas” is directed substances at their gaseous state, that continually flows under an applied shear stress, or external force. In some embodiments, the terms “gas” and “fluids” may interchangeably be used. In some embodiments, the term gas encompassed any type of pressurized gas capable of being stored and released from a closed container/chamber/tank. As used herein, a gas may include pure gas (such as, for example, pure oxygen), a mixture of gases (such as, for example, air), and/or a suspension of fine liquid droplets or fine solid particles in gas (for example, in the form of aerosol). Each possibility is a separate embodiment. In some exemplary embodiments, the gas is selected from, but not limited to: oxygen (O₂), nitrogen (N₂), carbon dioxide (CO₂), Aragon (Ar), Helium (He), air or any combination thereof. Each possibility is a separate embodiment.

The term Forced Vital Capacity (“FVC”) relates to the volume of air in the lung upon a deep breath. In some embodiments, the volume of air is measured in units of Liter(s).

The term Residual Volume and Forced Residual Volume (“RV/FRV”) relates to the volume of air in the lung after forced exhale or a cough. In some embodiments, the Residual Volume or Forced Residual Volume are measured in units of Liter(s).

The term Forced Expiratory Volume (“FEV1”) relates to the volume of air, forcefully exerted from, the lungs in a period of one second.

The term “PEF-PIF” relates to the difference between the peak expiratory airflow and peak inspiratory airflow.

The term “PIF/PEF” relates to the ratio between the peak inspiratory airflow and the peak expiratory airflow.

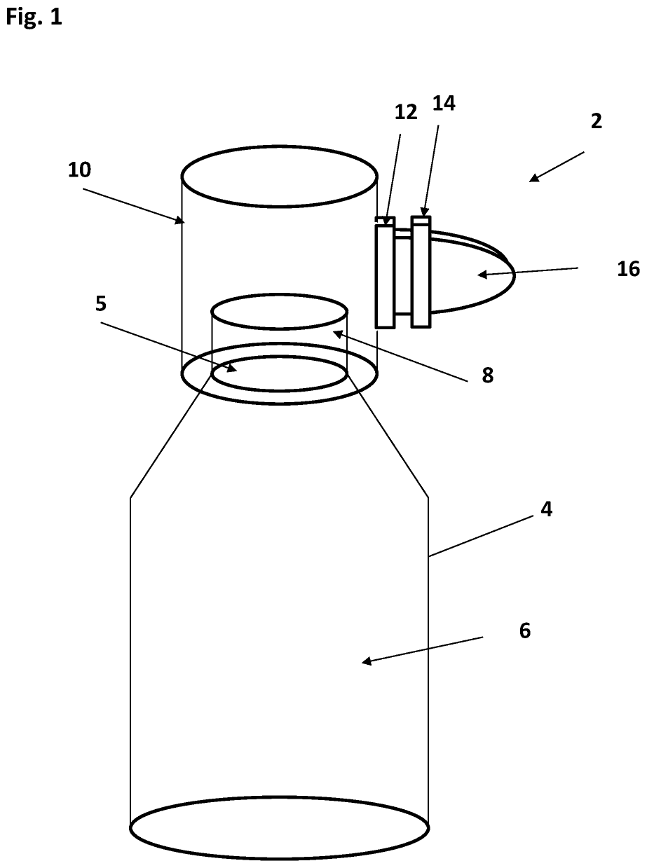

Reference is now made to FIG. 1, which schematically illustrates a front perspective view of device for delivering pressurized gas via, for example, inhalation by a subject, according to some embodiments. As shown in FIG. 1, device (2), includes a closed chamber/container/tank (shown as tank 4), capable of storing a reservoir of pressurized gas, such as, air (shown as reservoir 6). The container has an opening at the top region thereof (5), which can allow transfer of gas to and from the gas reservoir (6) that is confined within tank (4). The opening at the top end of the container may be sealed/covered by a top cover (shown as cover 8), which can be used to close/seal/cover the opening, so as to control the gas flow into and out of the gas reservoir and/or filling or emptying/depleting the reservoir. On the top region of the container, a delivery regulator (shown as regulator 10) is situated. The regulator may include one or more functional elements and/or adaptors, allowing its operation in regulating the passage of gas to/from the gas reservoir. For example, the regulator may include an adaptor, 12 that may be used to connect the regulator to external means capable of ultimately accessing the subject's airways. In some embodiments, such means may include any suitable tube or mask, such as, but not limited to: pharyngeal mask, laryngeal mask, face mask, endotracheal tube, and the like. For example, adaptor (12) shown in FIG. 1, can be used to connect to a pharyngeal mask adaptor (shown as adaptor 14). The external pharyngeal mask (16) represented in FIG. 1 is shown in the form of a mouthpiece (16), that can be used for securing a sealed route for the gas to the patient's lungs by insufflation/exsufflation, as further detailed below.

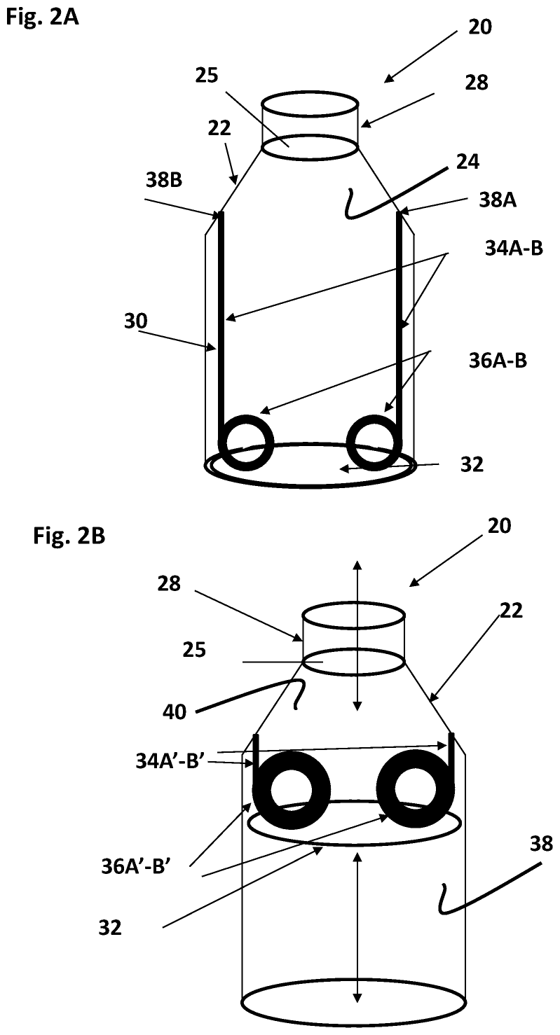

Reference is now made to FIGS. 2A-B, which schematically depict mechanism of storing and delivering pressurized gas under a constant or predetermined gradient of pressure, according to some embodiments. Shown in FIG. 2A is a longitudinal cross section of container/chamber/tank 20, which has an external wall/shell (wall 22). The wall of the container is preferably rigid and is able to withstand high pressures. The shell of the container defines an internal gas reservoir volume (shown as reservoir 24). The container has an opening at the top region thereof (25), which can allow transfer of gas to and from the gas reservoir (24) that is confined within chamber (20). The opening at the top end of the container may be sealed/covered by a top cover (shown as cover/cap 28), which can be used to close/seal/cover the opening, so as to allow the gas flow into and out of the gas reservoir and/or filling or emptying/depleting the reservoir. Further shown in the internal volume of the container, is pressure maintaining element, 30. Pressure maintaining element 30 includes a floating/movable floor, shown as, movable floor 32. Movable floor 32 can move along a longitudinal (vertical) axis, from the bottom region of the container to an upper region of the container. The dimension of movable floor 32, are preferably such that they coincide to the internal dimensions of the container, to thereby create a seal with the verticals walls of the container, such that no leak of gas occurs between the floor circumference and the internal walls of the container. By the upward/downward movement of movable floor 32, the pressure and/or volume of gas reservoir 24 may be maintained/altered, by decreasing/increasing the region (volume) defined between the movable floor and the upper end of the container (i.e., defining the reservoir). Pressure maintaining element 30 further includes means to change the vertical position/location/height of the movable floor within the container. The exemplary means to change to vertical position of the movable floor, may include any type of cables/springs/strings (shown as exemplary spring motors 34A-B), that may change in length and be collected/dispensed to/from corresponding storing elements (shown as storing drums 36A-B), that are further connected/attached/formed with movable floor (32). The cables/springs may further be connected/attached to the internal walls of the container, at an upper region of the container (for example, at regions 38A-B). The cables may be made of flexible or elastic material that may change its tension/strength/flexibility/compression/stretchiness. In some embodiments, the cables are made of rubber, plastic, metal, polymers, carbon or any materials known to those skilled in the art, or any combination thereof. Reference is now made to FIG. 2B, which shows a schematic cross section of a container, showing the Pressure maintaining element at a state after at least part of the gas has been dispensed (delivered). As shown in FIG. 2B, movable floor 32 has moved up in a vertical direction of container 20. Movable floor has moved upwards, via the pulling/collapsing/compressing/stretching of the strings/cables/springs (34A′-B′), relative to their resting position (shown in FIG. 2A (34A-B)). The pulled strings, which are now in a more condensed state are collected in drums 36A′-B′. As shown in FIG. 2B, when the movable floor is moving upward, the volume between the floor and the upper region of the container is reduced (shown as gas reservoir 40). Consequently, the pressure of gas contained in the reservoir is increased, decreased or maintained according to a predetermined pattern created by the cables/springs/strings and directed towards the upper opening of the container. The void volume (i.e. the volume where no gas is found, below the surface of the floating floor (shown as void volume 38), is increased as the floor moves vertically towards the upper end of the container.

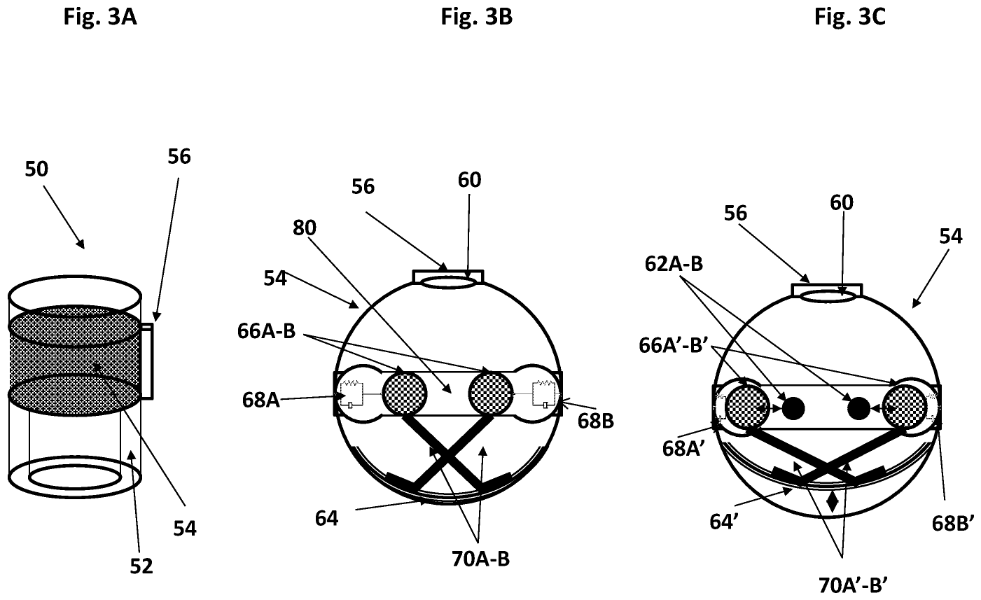

Reference is now made to FIGS. 3A-C which schematically depict a mechanism of a pressurized gas delivery regulator (pressure and/or flow regulating element), configured to deliver pressurized gas via insufflation upon inhalation by a subject, according to some embodiments. Shown in FIG. 3A is a schematic illustration of a perspective side view of an exemplary pressure and/or flow regulating element (delivery regulator) used for regulating passage of pressurized gas through a valve. Regulator (50), is configured to be placed or situated on a top region of a pressurized gas container of a device for delivering pressurized gas via (as illustrated in details in FIG. 1, above) to regulate the passage of pressurized gas from/to the container. The regulator may be placed on the opening of the container, directly, or via an adapter, shown as adaptor 52 in FIG. 3A. The regulator further includes a valve (54) and further adaptor (56) configured to connect the regulator to external means capable of directly or indirectly accessing the subject's airways.

Reference is now made to FIGS. 3B-3C which show schematic illustrations of a cross section of a valve unit of a pressurized gas delivery regulator, for regulating passage of pressurized gas in a closed mode (FIG. 3B) and open mode (FIG. 3C), wherein the valve is activated by inhalation of a subject. As shown in FIG. 3B, valve unit 54 includes opening/channels (shown as channel 60), which ultimately allows passage of gas from the gas container/tank (not shown), via adaptor 56, to external means capable of directly or indirectly accessing the subject's airways. Further included with the valve is deformable membrane/flexible wall (shown as membrane 64 in FIG. 3B). Membrane 64 is positioned at a distal region, opposing opening 60 (i.e., opposing the side that can connect to the external means capable of directly or indirectly accessing the subject's airways). Deformable membrane 60 may change its shape/tension, as further detailed below. The membrane may be connected/attached/formed with one or more points/regions on valve body/valve walls and to one or more levers/handles, shown as levers 70A-B. Levers 70A-B are further connected on their distal end to covers/caps (shown as covers 66A-B), which are situated on a rail (shown as rail 80), allowing their movement along the rail. The valve further includes resistance units (shown as resistance units 68A-B), which are connected to the valve body on one end and to covers/caps (66A-B) on the opposing end thereof. The resistance units are preferentially located on rail 80, or in close proximity thereto. The resistance units may be composed by one or more resistance components, such as, but not limited to: springs, dashpots, electrical resistance units, or any suitable type of resistance units, configured to resist a change in their formation/tension and return to their previous state after they have been deformed/contracted. Each possibility is a separate embodiment. Reference is now made to FIG. 3C, which shows valve unit 54 in an open state, which allows transfer of gas from the pressurized gas container (not shown), via opening/channels (shown as channel 60), through adaptor 56, to external means capable of directly or indirectly accessing the subject's airways. As shown in FIG. 3C, after the deformable membrane has been deformed (changed its shape, shown as deformable membrane 64′), to move away from the valve body walls, levers 70A-B (of FIG. 3A) have changed their position (shown in position 70A-′-B′ in FIG. 3C). Consequently, the movement of the levers results in the movement of covers/caps (66A′-B′) along rail 80, such that channels/openings 62A-B, that were covered/blocked/sealed by caps 66A-B (in FIG. 3B), are now open. Opening of these channels (62A-B), by the movement of the caps (66A′-B′) allows the flow of pressurized gas from the pressurized gas container (not shown) through the open channels (62A-B), ultimately to the external means capable of directly or indirectly accessing the subject's airways (via one or more adaptors, as detailed above). As further shown in FIG. 3C, the resistance unit are compressed/deformed/collapsed (shown as compressed resistance units 68A-′-B′). According to some embodiments, the resistance units are configured to limit the movement of the caps, such that upon the movement of the caps, the resistance units attempt to return to their previous mode, causing movement of the caps at a defined rate/duration along the rail, back to their previous location, to cover/seal channels 62A-B, to thereby function as open/close modes of the valve. In some embodiments, the deformation of the membrane is induced by inhalation of a subject, via the external means capable of accessing the subject's airways. When inhaling, the membrane is deformed, consequently, opening the caps and allowing gas passage from the pressurized gas container to the subject's airways, to thereby cause insufflation and induce an insufflation-exsufflation cycle.

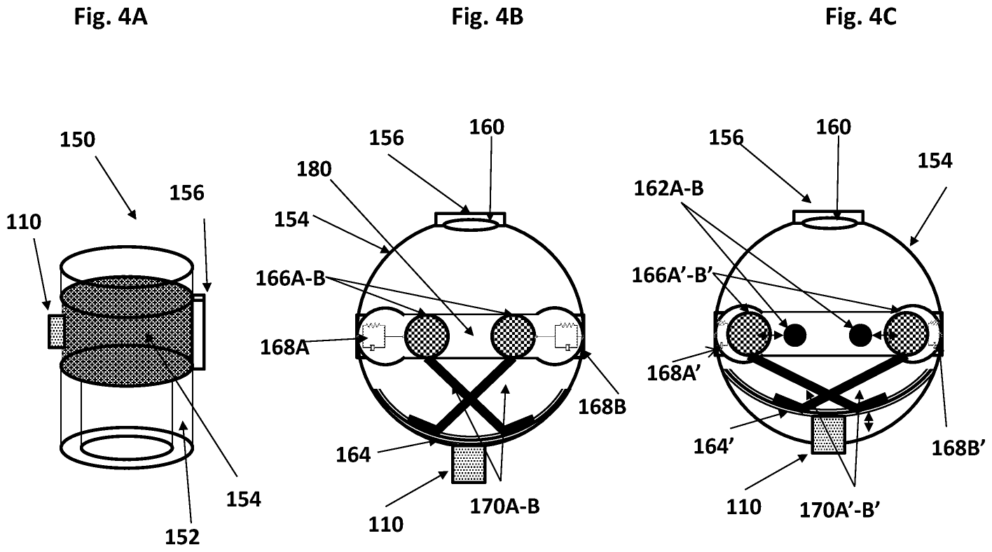

Reference is now made to FIGS. 4A-C which schematically depict a mechanism of a gas delivery regulator, configured to deliver pressurized gas via manual activation by a subject, according to some embodiments. According to some embodiments, the mechanism illustrated in FIGS. 4A-C resembles the mechanism illustrated in FIGS. 3A-C, however, the activation of the mechanism (i.e., the deformation of the membrane) is achieved by manually deforming the membrane, using a dedicated activation button, which is connected to or associated with the deformable membrane 164. By pressing or otherwise manipulating the activation button, the membrane is deformed, to activate the delivery mechanism (i.e., activate the valve).

Shown in FIG. 4A is a schematic illustration of a perspective side view of an exemplary delivery regulator (pressure and/or flow regulating element) used for regulating passage of pressurized gas through a valve. Regulator (150), is configured to be placed or situated on a top region of a pressurized gas container of a device for delivering pressurized gas via (as illustrated in details in FIG. 1, above) to regulate the passage of pressurized gas from/to the container. The regulator may be placed on the opening of the container, directly, or via an adapter, shown as adaptor 152 in FIG. 4A. The regulator further includes a valve unit (154) and optionally further adaptor (156) configured to connect the regulator to external means capable of directly or indirectly accessing the subject's airways. Further included is activation button (shown as button 110).

Reference is now made to FIGS. 4B-4C which show schematic illustrations of a cross section of a valve of delivery regulator, for regulating passage of pressurized gas in a closed mode (FIG. 4B) and open mode (FIG. 4C), wherein the valve unit is activated by manual manipulation (pressing) of activation button (Button 110), by a user. As shown in FIG. 4B, valve unit 154 includes an opening/channels (shown as channel 160), which ultimately allows passage of gas from the gas container (not shown), via adaptor 156, to external means capable of directly or indirectly accessing the subject's airways. Further included with the valve is deformable membrane/flexible wall (shown as membrane 164 in FIG. 4B). Membrane 164 is positioned at a distal region, opposing opening 160 (i.e., opposing the side that can connect to the external means capable of directly or indirectly accessing the subject's airways). Deformable membrane 160 may change its shape/tension, as further detailed below, by manually deforming the membrane, via an activation button, to which it is attached/connected with. The membrane may be connected/attached/formed with the valve body at one or more points/regions and to one or more levers/handles, shown as levers 170A-B. Levers 170A-B are further connected on their distal end to covers/caps (shown as covers 166A-B), which are situated on a rail (shown as rail 180), allowing their movement along the rail. The valve further includes resistance units (shown as resistance units 168A-B), which are connected to the valve body on one end and to covers (166A-B) on the opposing end thereof. The resistance units are preferentially located on rail 180, or in close proximity thereto. The resistance units may be composed by one or more resistance components, such as, but not limited to: springs, dashpots (for example, dampers which can resist the motion via viscous friction), electrical resistance units, or any suitable type of resistance units, configured to resist a change in their formation/tension and return to their previous state after they have been deformed/contracted. Reference is now made to FIG. 4C, which shows valve 154 in an open state, which allows transfer of fluid from the pressurized fluid container (not shown), via opening/channels (shown as channel 160), through adaptor 156, to external means capable of directly or indirectly accessing the subject's airways. As shown in FIG. 4C, after the deformable membrane has been deformed (changed its shape, shown as deformable membrane 164′), to move away from the valve body walls, levers 170A-B (of FIG. 4A) have changed their position (shown as position 170A-′-B′ in FIG. 4C). Consequently, the movement of the levers results in the movement of covers/caps (166A′-B′) along rail 180, such that channels/openings 162A-B, that were covered/blocked/sealed by caps 166A-B (in FIG. 4B), are now open. Opening of these channels (162A-B), by the movement of the caps (166A′-B′) allows the flow of pressurized fluid from the pressurized fluid container (not shown) through the open channels (162A-B), ultimately to the external means capable of directly or indirectly accessing the subject's airways (via one or more adaptors, as detailed above). As further shown in FIG. 4C, the resistance unit are compressed/deformed/collapsed (shown as compressed resistance units 168A-′-B′). In some embodiments, the resistance units are configured to limit the movement of the caps, such that upon the movement of the caps, the resistance units attempt to return to their previous mode, causing movement of the caps at a defined rate/duration along the rail, back to their previous location, to cover/seal channels 162A-B, to thereby function as open/close modes of the valve. In some embodiments, the deformation the membrane is induced by pressing/activating/manipulating, activation button, 110, which is connected or associated with the membrane, at the distal region thereof. When the button is activated, the membrane is deformed, detach from the valve walls and consequently, the respective caps are opened and gas passage from the pressurized gas container to the subject's airways is allowed.

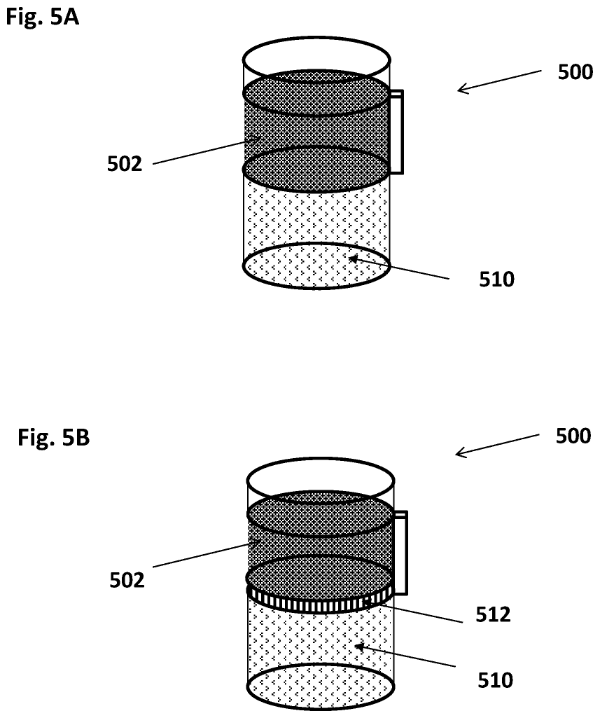

According to some embodiments, the pressure regulator illustrated in FIG. 3A-C or 4A-C, may further include aerosolisation chamber, that can aerosolize fluid or solid particles (the aerosol being any suitable solid or liquid that can be aerosolized, such as, for example, but not limited to: a drug solution, water, saline and any other solution or particles that could be aerosolized), stored within an aerosolisation chamber. The aerosolisation may be performed by perpendicular, swirling, jet, ultrasonic aerosolisation, or any other suitable method. In some embodiments, the aerosolisation chamber may include a particle filtering membrane (for example, a mesh or other suitable filter), to determine the particle size of the aerosolized particles delivered. Reference is now made to FIGS. 5A-B, which schematically depict mechanism of a perspective side view of an exemplary delivery regulator used for regulating passage of pressurized gas through a valve, and further having an aerosolisation chamber. As shown in FIG. 5A, pressure regulator (500), includes a valve (502) and aerosolisation chamber (510). As shown in FIG. 5B, pressure regulator (500), includes a valve (502), aerosolisation chamber (510) and filter membrane (512).

In some embodiments, the gas pressure and/or flow regulator, includes a chamber, which is a hollow body configured to facilitate equilibration. Such chamber is configured to fluidly connect between the pressurized gas container and the external means capable of accessing the subject's airways, via the various adaptors, as detailed above herein.

According to some embodiments, the chamber may also include a positive expiratory pressure (PEP) or a one-way valve to ensure one directional flow of air. In some embodiments, the pressure chamber may also include a safety valve, ensuring that the pressure introduced to the patient does not pass a set limit, based on the configuration and reservoir volume attached to the pressure regulator unit, for example, limiting the maximal pressure to, for example, 70 cmH2O, a recommended insufflation pressure for administration of 0.5-1 L of air. In some embodiments the chamber, valve or tank may further include an indicator presenting/showing the amount of stored gas or delivery units available for further use.

According to some embodiments, when hyperinflation is used to remove secretions, the tank and/or chamber can maintain constant or steady change in pressure to ensure PEF (PCF) of over about 160 L/min, PEF-PIF over about 20 L/min, and/or PIF/PEF bellow about 0.9. In some embodiments, the valve ensures predetermined insufflation time of about 1-6 seconds regardless to the user's inhalation, delivering a predetermined volume of about 0.5-2 L of gas in accordance to the user's lung compliance and physiology.

According to some embodiments, the gas pressure in the tank may be between about 10-50 atm.

According to some embodiments, the tank may include an expanded collapsible bag having a fixed maximum dimension and resizing capability, and wherein the bag is used for storing the gas, such that the bag volume defines the remaining effective/operational volume and the pressure change rate of the gas.

According to some embodiments, the tank's volume may change during insufflation to maintain a steady pressure or to control pressure changing rates. This control of pressure parameters may be obtained by using vacuum-induced force, a spring, a constant force spring or any other force inducing mechanism.

According to some embodiments, when long insufflation time is needed, the valve may not include or may not induce a closing (shut-off) mechanism, thereby delivering the entire gas content of the tank at once.

According to some embodiments, when multi-use is needed, the valve can include a spring or spring-like component to stop insufflation. According to some embodiment, when long insufflation time is needed for multi-use, a spring and dashpot dashpot (any suitable dashpot arrangement) or any other damping components may be used. Each possibility is a separate embodiment.

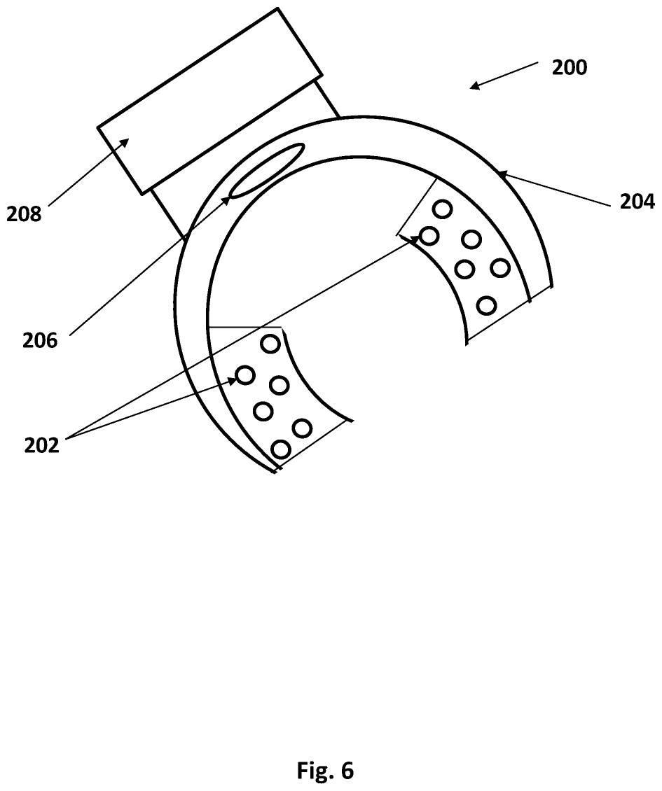

Reference is now made to FIG. 6, which schematically depicts a perspective view of an exemplary design of a pharyngeal mask, according to some embodiments. As shown in FIG. 6, a mask, embodied as a pharyngeal mask 200, which is a mouthpiece used for securing a fluidly sealed route for the gas from the pressurized gas container device to the patient's airways (in particular), the patients lungs, by insufflation/exsufflation. As shown in FIG. 6, mouthpiece mask (200) has on one end an adapter, configured to connect/adapt/attach to a corresponding adaptor of a gas delivery regulator of a device for delivering pressurized gas (such as, for example, adaptor 14 in FIG. 1, or adaptor 56 in FIG. 3A), or any other suitable adaptor (as further detailed below). On the opposing end, the mask further includes a biting (securing) surface (202), which the user (patient) can bite/hold with his teeth or tongue, to secure the mask in the oral/pharyngeal cavity. The biting region (surface) is designed to fit the teeth of the subject and is thus curved, so that, by biting and gripping the biting surface, the mask can be secured in the subject's mouth. This design further allows using the mask minimal hand-assistance. Hence, the mask is configured for self-use, also among subjects suffering from low neuromotor capacity. The biting surface is further configured to facilitate a tight seal of the mask, with the aid of a sealing element (shown as sealing element 204), which once placed in the pharyngeal cavity, ensures a direct, uninterrupted path from of the passing of the gas from the pressurized gas container of the device, via the corresponding adaptors, via a dedicated channel/passage (shown as channel 206) on the mask to the subjects airways. Channel 206, located on the adaptor end of the mask, allows the uninterrupted, sealed passage of gas from the pressurized gas container, via the pressure regulator, via the respective adaptors on the pressure regulator and on the mask to the into the patient's airway, upon activation of the pressure regulator of device (for example, by inhalation, manual pressure or any other suitable activation route, as detailed herein). Thus, when the mask is placed and secured in the subject's mouth, the gas which eventually flows via opening/channel 206, is directly directed into the subject's mouth, to thereby substantially reduce the risk of leakage and, therefore, the desired gas volume is inhaled by the subject, and does not need to rely on distribution or diffusion of the gas in the mask. In some embodiments, this is particularly of importance when the gas includes a drug (for example, in the form of aerosol). In this manner, by providing a direct, secured, and sealed path for the drug directly to the patients mouth, the leakage of the drug is substantially reduced and the effective amount of the drug directly reaching its target tissue (for example, the lung) is increased, compared to other means of providing a drug via inhalation, which are affected by distribution and diffusion of the drug in other types of masks.

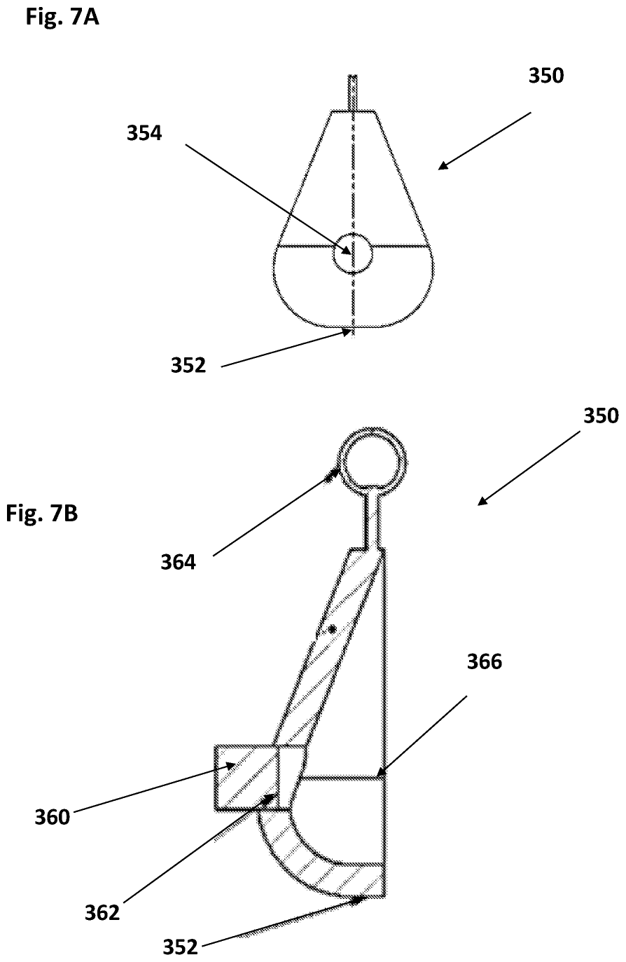

Reference is now made to FIGS. 7A-7B, which schematically depict a front view and a side view of exemplary a cup member of a mask, according to some embodiments.

FIG. 7A schematically depicts a front view of an exemplary cup member, 350, of a mask (such as, for example, a face mask). Cup member 350 is configured to fluidly connect to the pressurized gas container coupled at a distal end of the cup member, and optionally to the distal end of an adaptor (such as an adaptor of a mouth piece), at the proximal end of the cup member.

Since the gas is configured to be delivered directly, without any spacer, into the subject's mouth, the eyes of the patient will not be subjected to damage by the delivery of high volume of gas, or, a drug, if present within the gas, even if the mask is not completely sealed. The cup member may include a nose bridge (not shown) configured to seal the nose if needed. In some embodiments, the cup member may include a chin support assembly (352), located in a bottom section of the cup member, for mounting the mask on the subject's face. In some embodiments, the face mask may include or may be made of an elastic material that may be selected from, but not limited to: rubber, silicone, cloth, polyvinyl chloride (PVC), or any derivative or combination thereof.

In some embodiments, the facemask may contain one or more straps located in one of the outer surfaces of the mask which enables a user to place on the face without the use of digits. In some embodiments, the strap can be large enough for a wrist or a first or one or more digits to fit into the strap, upon doing so, the user can bring the mask upon the face without the usage of fine motor skills.

Reference is now made to FIG. 7B, which schematically depicts a side view of cup member 350 of FIG. 7A, used for placement against the face of the subject. Cup member 350 may include a PEP or a one-way membrane valve (360) and/or an optional filter (filter 362) positioned between the inner volume of cup member 350 and valve 360, and configured to prevent patient's secretions from blocking of valve 360. The cup member may further include a chin support assembly 352, an optional thumb loop 364, and a mouth-piece vector (366), configured to aid in placing the cup member in the mouth and/or to attach a suitable mouthpiece to be placed in the mouth of the patient. Thumb loop 364 is an optional assembly that is configured to provide comfortable access to the face mask and to enable easy wear of the mask.

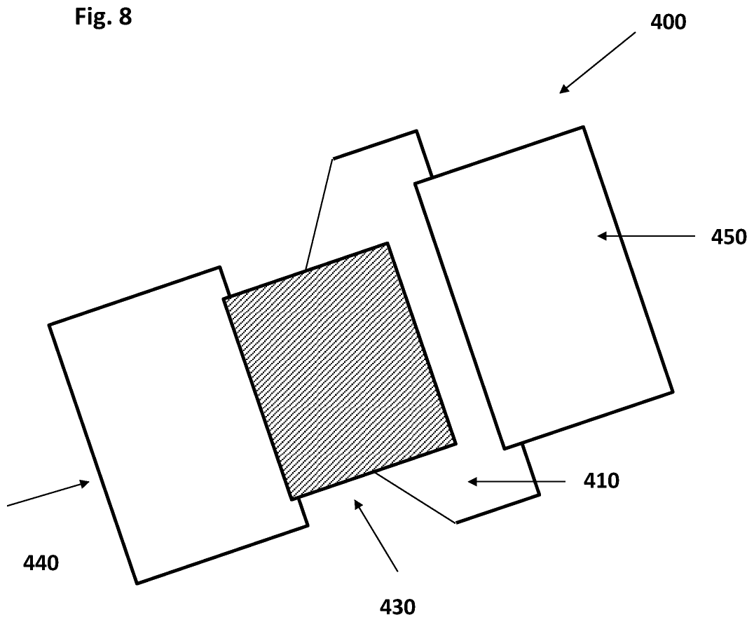

Reference is now made to FIG. 8, which in some embodiments is a schematic depiction of a connector, configured to connect to a cup member of a face mask and/or pharyngeal mask, which includes a valve. As shown in FIG. 8, connector (400) includes a valve (430), which can control/regulate/allow the gas connection between the cup member to a pressurized gas tank/container (not shown), positioned at a distal end (440) of the cup member (and optionally connected via suitable adaptors). Valve 430 may include a PEP or a one-way valve to prevent backflow and to allow deeper gas (for example, air or aerosol) delivery into the subject's lungs and improve hyperinflation capacity. This is beneficial in particular for SCI patients and may improve effectivity among subjects with high residual volume and/or low respiratory capacity of the lungs. Also shown in FIG. 8 is separation membrane (410) and chamber 450. In some embodiments, upon exhalation by a subject, membrane 410 can deform to seal valve 430, ultimately preventing the inhaled gas to be released to the pressurized gas tank, from chamber 450. In some embodiments, chamber 450 comprises an elastic and biocompatible material selected from the group consisting of: polylactic acid (PLA), polypropylene (PP), or any combination thereof.

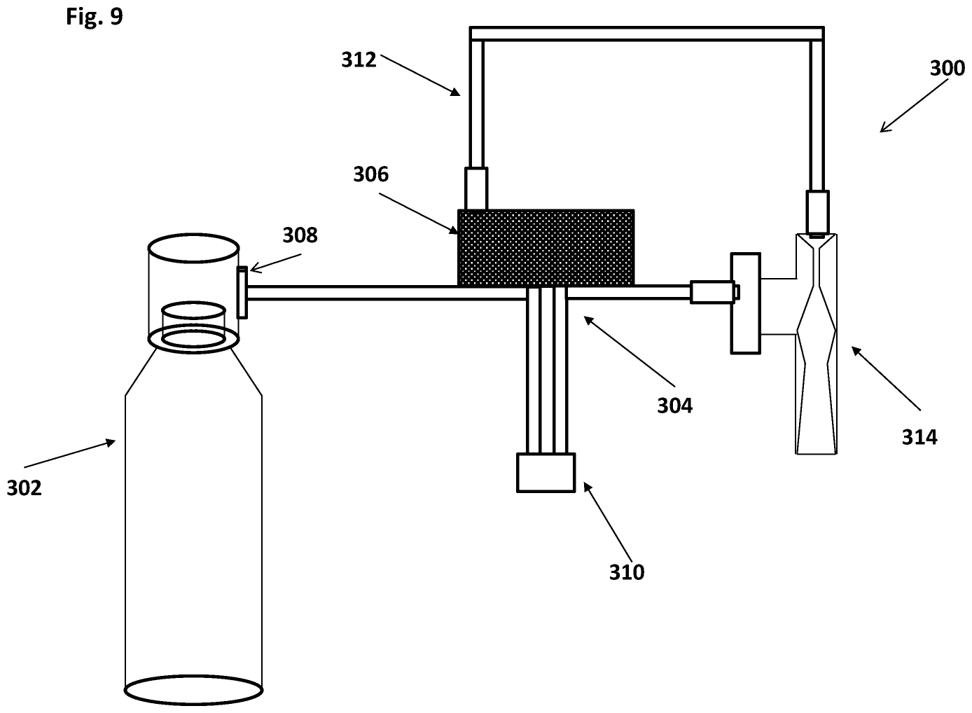

Reference is now made to FIG. 9, which schematically depicts an automatic system of delivering and extracting pressurized gas (insufflation-exsufflation) to a subject via a mask, according to some embodiments. As shown in FIG. 9, automatic system, 300, includes two units/devices that are functionally and/or physically connected or associated, to ultimately provide and extract gas to a subject's airways, via a suitable mask. System 300 includes two units, a first unit connected to the pressurized gas device (302), via regulator (306) to an insufflation port (304; left) and a second unit connected to venturi-based device (314), via an exsufflation port (304; right). The system further includes a pressure sensor (not shown) imbedded in the pressure regulator (306) (for example, in the form of an electrical pressure regulator), for allowing fluid gas from the pressurized gas device (302) to a mask of the subject, via an adaptor, 310 that connects/adapts/attaches to the mask airway. According to some embodiments, insufflation is triggered by inhalation (as detected by a sensor imbedded in regulator 306) and once inhalation is completed and exhalation (cough) begins, exsufflation is assisted as follows: gas flow from the pressurized gas device (302) is directed through a bypass (shown as bypass 312), into venturi device (314), thus creating negative pressure resulting in extraction of gas from the exsufflation port (304; right), in accordance with the Bernoulli law. According to some embodiments, by varying the ratios of the chambers in the pressure changing device (314), it can be adapted to fit various ranges of negative pressures, extraction rates of the fluid and/or fluid volumes dispensed. According to some embodiments, exsufflation is triggered by exhalation (cough; as detected by a sensor imbedded in regulator 306).

According to some embodiments, using the platform disclosed herein, enables insufflation of 0.5-3 L of gas (such as, air), in a single administration, to the pulmonary system from the pressurized gas tank, and in some embodiments, exsufflation of the same, higher or lower volume of gas. Throughout this process, the device is able to assist coughing, increasing the FVC. In some embodiments, where exsufflation is enabled, the device also increases FEV1 and reduce FRV.

According to some embodiments, when utilized, the face mask may be used to absorb, wipe and/or store extracted mucus and other pulmonary disturbances.

In some embodiments, after insufflation, air may be redirected by the patient to induce an effective sneeze or nose blowing, clearing the nasal airways and the nasal cavity. Such actions are limited or unable to be performed effectively by SCI and other neuromuscular deficient patients, for similar reasons to ineffective coughing described above. Accordingly, utilizing the disclosed platform can allow, in some embodiments, for secretions to be collected or absorbed by the respiratory mask.

In some embodiments, the methods, devices and systems, may be personalized, adapted and repeatedly used for multiple times. In some embodiments the methods, devices and systems, may be meant for a single-use. In some embodiments, the mask is configured for a single use, even if the other components (such as, pressurized gas tank, regulators, etc.), are multiplicity used.

According to some embodiments, the gas (such as air and/or aerosol) is administered directly into the subject's respiratory system and delivered to the lungs, including lower and upper respiratory tracts and pulmonary alveoli. In some embodiments, such as an active targeted drug delivery decreases the effective dose required, by reducing drug loss on the way to the target site to thus reduce adverse effects or to enable the use of other drugs with an active site that is unlikely to be accessed in other methods of administration. According to further embodiments, avoiding the gastrointestinal tract and a systemic drug delivery is generally beneficial and in particular for patients suffering from a neuromuscular deficiency. The active delivery of a large volume of pressurized gas and/or drug directly to the lungs is advantageous particularly to subjects having high residual volume of air in the lungs and/or low respiratory capacity.

According to some embodiments, the gas (such as air and/or aerosol) is administered in a cumulative manner, which is advantageous for delivery of a pulmonary drug having an immediate effect or indications of effectiveness, e.g., Ventolin or capsaicin. Ventolin is a short-term bronchodilator used also for treating acute asthma episodes. Using the platform for cumulative dosing of Ventolin enables the subject to stop inhaling the drug when the episode is over. This provides, in some embodiments, individualization of drug dosage or personalized drug dosage according to immediate response. This may further reduce adverse effects and insensitivity and overdosing events, since patients can adjust drug dosage by halting/stopping administration upon achieving a desired effect.

According to some embodiments, the platforms disclosed herein can be used in targeted delivery of the drug to a specific target site/region/tissue in the subject airways, by adjusting the pressure parameters and/or the particle size of the drug.

In some embodiments, the drug may be selected from, but not limited to: a chemotherapeutical drug, anti-cancer drug, anti-inflammatory drug, a corticosteroid, a respiratory drug, a cough inducer, an anti-microbial drug, an anti-viral drug, an anti-fungal drug, or any combination thereof. Each possibility is a separate embodiment.

In some embodiments, the drug includes a bronchodilator. In some embodiments, the drug includes Ventolin. In some embodiments, the drug includes Capsaicin.

In some embodiments, Capsaicin is provided to induce coughing. Capsaicin-induced coughing may be used to clear respiratory secretions and/or for diagnosis of respiratory compromisation of patients having a nervous system disorder or injury, such as central nervous system (CNS) disorders, cervical injuries and/or spinal cord injuries (SCI). The concentration of capsaicin used to induce coughing indicates the respiratory involvement of the disorder or severity of injury. Furthermore, when the subject coughs, further delivery of drug ceases thus prevents a drug overdose.

In some embodiments, the drug is used for treating respiratory infections (e.g., influenza, pneumonia) or excessive secretions (e.g., mucus), difficulty in breathing (e.g., asthma), bronchospasm, bronchiectasis, chronic obstructive pulmonary disease (COPD), or any combination thereof.

In some embodiments, the pressurized gas tank reservoir is disposable, hand-held, self-managed and mounted onto the mask. In such embodiments the volume is of about 50-500 mL and total weight under about 300 g.

In some embodiments, the pressurized gas tank reservoir is designed for multi-use, refillable and may be connected indirectly onto the mask. At such embodiments, a volume is of about 1-20 L is capable of being placed/mounted onto a wheelchair or any other transportation device supporting SCI or other neuromuscular deficient patients.

In some embodiments, the pressurized gas may be selected from, but not limited to: oxygen (O₂), nitrogen (N₂), carbon dioxide (CO₂), Aragon (Ar), Helium (He), air or any combination thereof. Each possibility is a separate embodiment. In some embodiments, the pressurized gas may be stored in a tank (container), as detailed above. In some embodiments, the tank stores a positively pressurized gas. In some embodiments, the gas pressure in the reservoir is about 10-30 atm, not more than about 50 atm. In some embodiments, the gas pressure in the reservoir is about 50-250 atm/not more than about 300 atm.

In some embodiments, the gas pressure in the gas reservoir (in the container/tank) is about 5-300 atm, or any subranges thereof. In some embodiments, the gas pressure in the gas reservoir (in the container/tank) is about 5-270 atm. In some embodiments, the gas pressure in the gas reservoir (in the container/tank) is about 10-250 atm. In some embodiments, the gas pressure in the gas reservoir (in the container/tank) is about 10-50 atm. In some embodiments, the gas pressure in the gas reservoir (in the container/tank) is about 25-300 atm. In some embodiments, the gas pressure in the gas reservoir (in the container/tank) is about 30-280 atm. In some embodiments, the gas pressure in the gas reservoir (in the container/tank) is about 40-260 atm. In some embodiments, the gas pressure in the gas reservoir (in the container/tank) is about 50-250 atm.

As detailed above, on average, healthy subjects, capable of coughing effectively has a FVC of 4-5 L, a FEV1 of 3-4 L/s and a FRV of 1-2 L. SCI patients with tetraplegia (high injury, associated with higher risk and incidence of respiratory infections) has a 40-70% of the FVC, 45-75% of the FEV1 and 110-160% of the FRV of an uninjured patient. Essentially, this means that an SCI patient exhale approximately 17% the air volume of a healthy patient (0.5 L in comparison to the 3 L exhaled by an uninjured subject) at a much lower speed, producing a significantly lower shear-force on pulmonary disturbances and producing an ineffective cough. Even patients with paraplegia, which has better pulmonary functions, fall as low as 70% FVC and FEV1 and up to 150% FRV exhaling less than a half than an uninjured subject would, at a lower speed, producing an insufficient or ineffective cough. Therefore, the disclosed devices and systems can by adjusted to function on the range of volume and pressure parameters of an SCI patient and an uninjured patient described herein in order to accomplish optimal coughing based on these parameters. In addition, in some embodiments these parameters may be adjusted manually based on the optimal results and preference of the patient. In some embodiments, the parameters of the individual patient can be diagnosed/determined automatically by the devices and systems (platforms), by introducing pressure and volume sensors or response elements as is known in the art to automatically readjust the parameters of pressure and volume for inhalation of air into the lungs and/or the rapid exhalation of air to induce cough.

According to some embodiments, utilizing the platforms disclosed herein (including, devices, systems, kits, valve-units, and the like), allows the advantageous delivery of continuous fluid (air) flow, in particular, even if large volumes are delivered. For example, the volumes may be in the range of about, 0.25-5 L, or any subranges thereof. For example, the volumes may be in the range of about, 0.5-4 L, or any subranges thereof. For example, the volumes may be in the range of about, 0.75-3 L, or any subranges thereof. For example, the volumes may be in the range of about, 1-2 L, and any subranges thereof.

In one embodiment, the drug may be administered to the subject in the form of an aerosol. In some embodiments, the mask (for example, face mask and/or pharyngeal mask) is disposable and configured for a single use.

According to some embodiments, a face mask is provided for delivering pressurized gas (for example air) to a subject, the mask comprising: a cup member having a peripheral edge for placement against the face of the subject, said cup member is configured to fluidly connect to a pressurized gas tank; and a valve configured to controllably fluidly connect the gas tank to said cup member, wherein, when said mask is placed on the subject's face and the gas tank is connected to the cup member, inhalation by the subject causes said valve to open and gas to be released from the gas tank and forced into the cup member and into the subject's mouth.

According to some embodiments, a system is provided for insufflation and exsufflation of pressurized gas to a subject, the system comprising: a face mask comprising a cup member having a peripheral edge for placement against the face of the subject, said cup member is configured to fluidly connect to a pressurized gas tank via an insufflation port and to a venturi-based device via an exsufflation port; and a embedded sensor functionally associated with a regulator configured to trigger, upon the beginning of inhalation of the subject, insufflation of pressurized gas from the pressurized gas tank via the insufflation port and to the cup member, wherein the system is further configured to induce exsufflation after a predetermined time or upon exhalation (coughing) detected by the embedded sensor of a volume of the pressurized gas by directing the gas flow to the venturi-based device.

According to some embodiments, the mask may further include a membrane located between the mouth and the valve when said mask is worn, and wherein inhalation by the subject, causes deformation of said membrane, which causes said valve to open. The mask may further include a mouthpiece for securing said mask to said mouth. The mouthpiece may include a bite surface for securing said mask by the teeth of the subject.

According to some embodiments, the pressurized gas may include a drug (e.g., aerosolized drug). The drug may be configured for administration to the respiratory system of the subject. The drug may be used for treating respiratory infections, excessive secretions, asthma, bronchospasm, bronchiectasis, chronic obstructive pulmonary disease (COPD), lung cancer or other abnormal pulmonary conditions, or any combination thereof. The drug may be selected from a group consisting of: an anti-inflammatory drug, a corticosteroid, a respiratory drug, a cough inducing drug, an anti-microbial drug, an anti-viral drug, an anti-fungi drug, cannabinoids, immunotherapy, chemotherapy or any other substances that may be delivered via the pulmonary route and any combination thereof. According to some embodiments, the drug may include capsaicin or any other cough inducing agent. According to some embodiments, the drug may be used for inducing coughing in a subject. According to some embodiments, the mask (e.g., for administering a drug) may be used for inducing coughing in a subject suffering from spinal cord injury (SCI). According to some embodiments, the mask may be configured to stop drug delivery once a cough is induced. According to some embodiments, the mask may include a pressure and/or volume sensor or responding elements and a regulator. The sensor/response element may be configured to activate the regulator upon inhalation to allow gas flow from the gas tank to the mouthpiece during inhalation. According to some embodiments, the mask may further include a chamber, wherein the cup member is configured to fluidly connect to the pressurized gas tank via said chamber, and wherein said chamber is configured to facilitate pressure equilibration.

According to some embodiments, the valve may include a positive expiratory pressure (PEP) one-way valve configured to prevent backflow and thus to facilitate deeper drug delivery into the lungs and/or to increase hyperinflation capacity of the lungs.

According to some embodiments, the mask may further include a chin support assembly to facilitate self-mounting of the mask on the subject's face with minimal hand-assistance.

According to some embodiments, the mask may further include a nose bridge for sealing the nose when said mask is worn.

According to some embodiments, the mask may include an elastic material. According to some embodiments, the mask is disposable and/or recyclable.

According to some embodiments, there is provided a face mask for delivering pressurized gas (such as air) to a subject, the mask comprising: a cup member having a peripheral edge for placement against the face of the subject, said cup member is configured to fluidly connect to a pressurized gas container (tank); and a valve configured to controllably fluidly connect the gas container to said cup member, wherein, when said mask is placed on the subject's face and the gas tank is connected to the cup member, inhalation by the subject causes said valve to open and gas to be released from the fluid tank and forced into the cup member and into the subject's mouth.

According to some embodiments, the mask may further include a membrane located between the mouth and the valve when said mask is worn, and wherein inhalation by the subject, causes deformation of said membrane, which causes said valve to open. In some embodiments, the mask may further include a mouthpiece for securing said mask to said mouth. In some embodiments, the mouthpiece includes a bite surface for securing said mask by the teeth of the subject.

According to some embodiments, there is provided a one-way valve unit. In some embodiments, the one-way valve unit configured to controllably release pressurized gas from a closed pressurized fluid tank/reservoir, the valve unit comprising a deformable membrane configured to deform only upon inhalation by a subject, wherein upon deformation of the membrane, one or more gas passage(s) in the valve unit are at least partially opened, to allow the movement of the pressurized gas movement from to the pressurized gas tank; wherein the valve unit further comprises one or more resistance units, configured to resist the membrane deformation, and allow controlling the gas passages. In some embodiments, the resistance unit comprises one or more of: springs, dashpot, electrical resistant units, or any combination thereof. In some embodiments, the valve unit may be configured to maintain a constant change in the gas pressure, such that the difference between a peak expiratory airflow (PEF) and a peak inspiratory airflow (PIF) is over about 20 L/min and/or the ratio between the peak inspiratory airflow (PIF) and the peak expiratory airflow (PEF) is lower than about 0.9.

According to some embodiments, the platforms disclosed herein may be used to increase adherence for cough assisting, respiratory exercise or any combination thereof.

According to some embodiments, the platforms disclosed herein may be used to lower acute pulmonary infections rates, prevent acute pulmonary infections, lower acute pulmonary complication rates, prevent acute pulmonary complications, lower chronic pulmonary complication rates, prevent chronic pulmonary complications or any combination thereof.

According to some embodiments, the platforms disclosed herein may be used to treat acute pulmonary infections, shorten the duration of acute pulmonary infections, treat acute pulmonary complication, shorten the duration acute pulmonary complications, treat chronic pulmonary complication, shorten the duration chronic pulmonary complications or any combination thereof.

According to some embodiments, the platforms disclosed herein may be used at home, by the user (subject) alone, or with the help of a caregiver.

According to some embodiments, the platforms disclosed herein may be used in a clinical setting, by the user alone, or with the help of a caregiver/clinician.

According to some embodiments, the platforms disclosed herein may be stored, attached or mounted on a motion assisting device such as, but not limited to: walker, wheelchair, motorized chair, extra-skeleton or any combination thereof.

According to an aspect of some embodiments of the present disclosure, there is provided a kit for delivering pressurized gas to a subject. The kit comprises a face mask and a pressurized gas tank configured for use with the mask. The face mask includes a cup member and a valve.

According to some embodiments there is provided a kit for delivering pressurized gas to a subject, the kit comprising: a face mask; and a pressurized gas tank configured for use with said mask.

According to some embodiments, there is provided a method for delivering pressurized gas to a subject, the method includes: adjusting a face mask according to embodiments disclosed herein, to the subject's face and upon inhaling, triggering a release of pressurized gas from the gas tank such that the gas is forced into the cup member and into the subject's mouth.

In another embodiment, the present disclosure provides a method for delivering pressurized gas to a subject. The method comprises a step of adjusting a face mask to the subject face. The method comprises a step of opening the valve by inhaling, thereby releasing pressurized gas from the gas tank and forcing the gas into the cup member and into the subject's mouth.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1-67. (canceled)

68. A device for delivering pressurized gas to a subject, the device comprising:
a tank holding a reservoir of pressurized gas;
a pressure maintaining element located within the tank, wherein the pressure maintaining element comprises a moveable floor and a constant force element functionally connected to the movable floor and configured to exert a constant force thereon, such that when gas is expelled from the tank, the constant force element causes an elevation of the moveable floor, thereby reducing the volume of the tank so as to maintain an essentially constant pressure within the tank allowing delivery of the entire content of the gas in the tank at a constant pressure;
an asymmetric valve comprising
an insufflation activating element configured to directly or indirectly open the valve in coordination with the subject's inhalation, thereby releasing the pressurized gas from the pressurized gas reservoir to provide insufflation of the subject; and
one or more resistance units configured to resist the reclosing of the valve, thereby controlling the duration of the insufflation,
wherein the combined operation of the pressure maintaining element and the asymmetric valve ensure that the volume of pressurized gas delivered to the subject is a predetermined volume independent of the subject s inhalation volume, wherein the predetermined volume is in a range of 0.5-3 L; and
a first adaptor, fluidly connected to the valve, configured to fluidly connect to a corresponding adaptor of a mask configured to interact with the airways of the subject.

69. The device according to claim 68, wherein the insufflation activating element is configured to activate insufflation on response to inhalation by a subject, or in response to manual activation.

70. The device according to claim 68, wherein the valve unit comprises a coupling element, connecting the insufflation activating element to a sealing unit, the coupling element configured to move the sealing unit upon activation of the insufflation activating element, to thereby allow opening of gas passages located between the pressurized gas reservoir and the first adaptor, to allow pressurized gas movement from the pressurized gas reservoir, through the valve unit, to the first adaptor.

71. The device according to claim 68, wherein the duration of insufflation is over about 0.7 seconds.

72. The device according to claim 68, wherein the one or more resistance units are selected from: springs, dashpots and/or electrical resistance units.

73. The device according to claim 68, wherein the gas comprises a drug.

74. The device according to claim 68, wherein the tank further comprises an aerosol chamber configured to aerosolize the drug.

75. The device according to claim 68, wherein the aerosol chamber further comprises a particle size filter configured to determine the aerosolized particle size.

76. The device according to claim 68, wherein the constant force element comprises one or more of: cables, springs, strings, motors, and/or motion units.

77. The device according to claim 68, wherein the combined operation of the pressure maintaining element and the asymmetric valve ensure that the volume of pressurized gas delivered to the subject is about 100-600% of the user's inhalation capacity.

78. The device according to claim 68, wherein the combined operation of the pressure maintaining element and the asymmetric valve ensure that a peak expiratory airflow (PEF) or peak coughing flow (PCF) of the pressurized gas delivered to the subject is over about 160 L/min.

79. The device according to claim 68, wherein the combined operation of the pressure maintaining element and the asymmetric valve ensure that the difference between the peak expiratory airflow (PEF) and the peak inspiratory airflow (PIF) is over about 20 L/min and/or a ratio between the peak inspiratory airflow (PIF) and the peak expiratory airflow (PEF), is lower than about 0.9.

80. The device according to claim 68, further comprising a safety valve configured to ensure that the maximal pressure insufflated is lower than about 75 cmH2O.

81. The device of claim 68, wherein the gas pressure in the pressurized fluid tank is between about 10 to about 50 atm.

82. The device of claim 68, wherein the pressurized gas tank comprises an expanded collapsible bag having a fixed maximum dimension, and wherein the bag is used for storing the gas, such that the bag minimal volume defines the remaining effective or operational volume.

83. The device of claim 68, for use in inducing coughing in a subject suffering from spinal cord injury (SCI) or neuromuscular deficiencies.

84. The device of claim 68, wherein the mask is a mouthpiece.

85. The device of claim 68, wherein the mask is a facial mask.

86. A method for delivering a predetermined dose of a drug to a subject, the method comprising:
adjusting a mask to the subject's face; and
upon inhaling, triggering a release of a pressurized gas from a device for delivering pressurized gas to a subject, the device comprising:
a pressurized gas tank holding a reservoir of pressurized gas;
an aerosol chamber configured to aerosolize the drug;
a pressure maintaining element located within the tank, wherein the pressure maintaining element comprises a moveable floor and a constant force element functionally connected to the movable floor and configured to exert a constant force thereon, such that when gas is expelled from the tank, the constant force element causes an elevation of the moveable floor, thereby reducing the volume of the tank so as to maintain an essentially constant pressure within the tank allowing delivery of the entire content of the gas in the tank at a constant pressure;
an asymmetric valve comprising
an insufflation activating element configured to directly or indirectly open the valve in coordination with the subject's inhalation, thereby releasing the pressurized gas and the aerosolized drug to the subject; and
one or more resistance units configured to resist the reclosing of the valve, thereby controlling the duration of the insufflation,
wherein the combined operation of the pressure maintaining element and the asymmetric valve ensure that the volume of pressurized gas delivered to the subject is a predetermined volume independent of the subject s inhalation volume, wherein the predetermined volume is in a range of 0.5-3 L thereby delivering the predetermined dose of the drug to the subject.

87. The method of claim 86, wherein the methods allows one or more of: treating acute pulmonary infections, shorten the duration of acute pulmonary infections, treat acute pulmonary complication, shorten the duration acute pulmonary complications, treat chronic pulmonary complication, shorten the duration chronic pulmonary complications, or any combination thereof.
